(19) United States
(12) Patent Application Publication
(10) Pub. No.: US 2009/0306654 A1
(43) Pub. Date: Dec. 10, 2009

(54) DEVICE AND METHOD FOR THE CONTROLLED THERMAL ABLATION OF TUMORS BY MEANS OF HIGH-FREQUENCY ELECTROMAGNETIC ENERGY

(76) Inventor: Giberto Garbagnati, Milano (IT)

Correspondence Address: Steinfl & Bruno, 301 N Lake Ave Ste 810, Pasadena, CA, 91101, US

(21) Appl. No.: 12/295,218

(22) PCT Filed: Mar. 31, 2006
(86) PCT No.: PCT/IT2006/000211

§ 371 Date: Aug. 14, 2009

(51) Int. Cl. A61B 18/14

(52) U.S. Cl. 606/41

(57) ABSTRACT

A device for the thermal ablation (TA) by means of high frequency electromagnetic energy comprising a hollow element (1), one or more electrodes (1, 10) connected to an electromagnetic energy generator at high frequency, e.g. radiofrequencies or microwaves, said hollow element (1) being tightly inserted into an expandable membrane (3). A viscous and electric conductive substance (6) is injected into the membrane (3) through one or more openings (4) provided on the portion of the hollow element (1) being enclosed in said membrane. The invention also relates to a method for the TA by means of high frequency electromagnetic energy using the above-mentioned device.

The present invention relates to a device and a method for the treatment of tumors by means of thermal ablation (TA) induced by electromagnetic energy, e.g. in the radiofrequencies (RF) or in the microwaves (MW) range, and particularly to a device and a method for the TA which allows to obtain lesions having a large volume and a predictable and controllable shape.

It is known that the TA procedure induced by electromagnetic energy essentially consist of inserting into a tumoral mass an electrode that, being supplied with electromagnetic energy at a suitable frequency, leads to the generation of heat in the surrounding tumoral tissues, thus causing their coagulative necrosis. The electrode, being generally placed at the end of a needle or a catheter, is percutaneously introduced in the mass of the tumor and it is guided by means of echography or other visualization technique known in the art. This procedure has proved to be for the ablation of tumors of the liver and it has recently been suggested for the ablation of tumors of lung, kidney and other parenchymal organs.

One of the major problems of this kind of procedure consists of the difficulty of destroying tumoral masses having a diameter that is larger than 3 cm. The main reason is that the energy delivered through the electrode inserted in the tumoral mass can not be indefinitely increased. In fact, if on one hand the delivery of high power allows to increase the size of the thermal lesion, on the other hand it causes a rapid dehydration of the tissue being closest to the electrode with the consequent impossibility of delivering further energy to the surrounding tissue.

Another problem of the known art is that controlling the shape of the generated thermal lesion is not possible, resulting in the risk of generating thermal lesions poorly corresponding to the shape of the tumor.

Devices and methods for increasing the volume of the thermal lesion in the tumoral mass are already known, consisting of infusing a conductive liquid therein, which transmits energy all around due to its electric conductivity. For instance, U.S. Pat. No. 6,911,019 discloses a catheter provided with an helicoidal needle that is inserted in the tumoral mass in order to create an helicoidal cavity being infused with a conductive liquid. The object is to create a channel with a prescribed shape in order to control the size of the thermal lesion. However, this method has the drawback of generating a thermal lesion having an irregular shape and a volume being difficult to predict due to the uncontrollable distribution of the conductive liquid into the tissues.

In patent application US 20040006336 a device is disclosed showing a hollow electrode that allows to improve the infusion of the conductive liquid into the tissue. Also this device exhibits the drawback of not allowing the control of the distribution of the conductive liquid into the tissues, that is the size of the zone being subject to the TA.

Object of the present invention is thus to provide a device and a method for the TA being free from the above-mentioned drawbacks, being suitable for increasing the volume of the thermal lesion to the utmost and being suitable for giving it a shape that is as round as possible. Such an object is achieved with the device for TA according to the present invention, whose characteristics are specified in claim 1. Further characteristics of such a device are specified in the dependent claims. In the subsequent claims the characteristics of the method for TA according to the present invention are specified.

Thanks to the use of a substance being electrically conductive, keeping the tissue hydrated around the active part of the electrode and the impedance of the system constantly low, combined with the use of an expandable membrane, locally pressing the tissues to be treated, it is possible to transfer an adequate power level to the tumoral tissue wherein the electrode is inserted, for a much longer time without being limited by the dehydration of the tissues surrounding the electrode.

One advantage of the device and the method for the TA according to the present invention, is that the shape and the volume of the generated thermal lesions are regular and predictable in an extremely precise way. In fact the above-mentioned substance is injected inside a semi-permeable and expandable membrane and closely contacts the tissues surrounding the device while remaining enclosed in the known volume of the membrane.

Another advantage provided by the device and the method for the TA according to the present invention is that the extraction of the device from the thermal lesion is facilitated by leaving the bulkiest part, i.e. the expandable membrane, “in situ” thus remarkably simplifying the operation.

A further advantage of the device and the method for the TA is that they are usable with electromagnetic energy both in the radiofrequencies and the microwaves range, with little manufacturing differences which will be promptly evident to those skilled in the art.

This and other advantages of the device for TA according to the present invention will be evident to those skilled in the art from the following detailed description of some embodiments thereof with reference to the annexed drawings wherein:

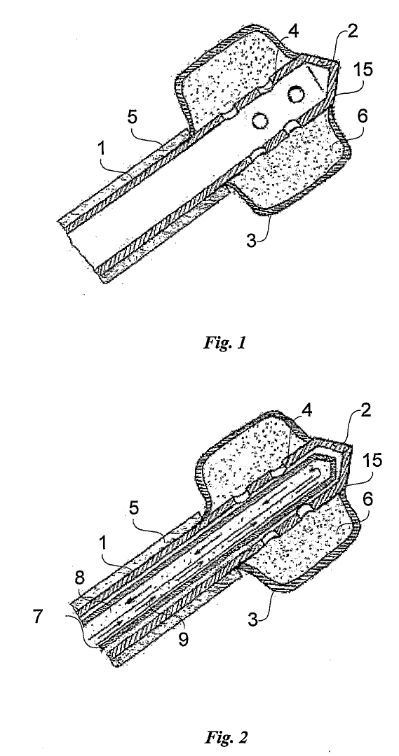

FIG. 1 shows a sectional detailed view of the end of the hollow element of one embodiment of the device for the TA;

FIG. 2 shows a sectional detailed view of the end of the hollow element of another embodiment of the device for the TA;

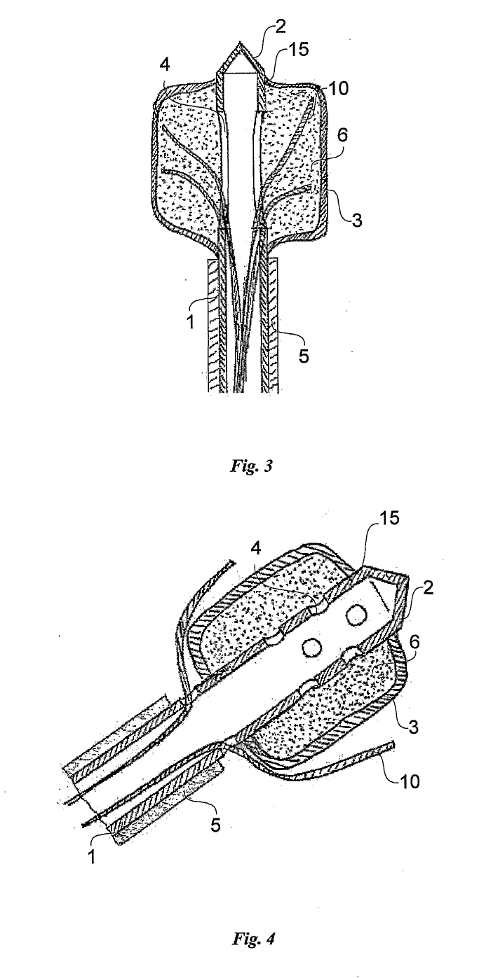

FIG. 3 shows a sectional detailed view of the end of the hollow element of another embodiment of the device for the TA;

FIG. 4 shows a sectional detailed view of the end of the hollow element of still another embodiment of the device for the TA;

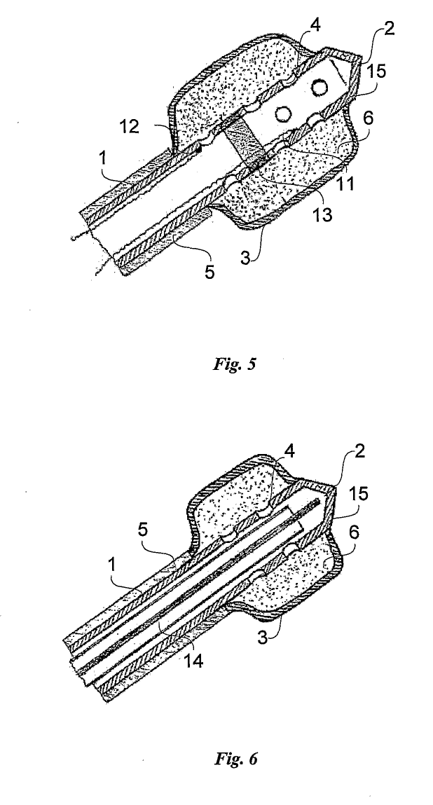

FIG. 5 shows a sectional detailed view of the end of the hollow element of a further embodiment of the device for the TA; and

FIG. 6 shows a sectional detailed view of the end of the hollow element of still a further embodiment of the device for the TA.

FIG. 1 shows cross section of the device for the TA according to one embodiment of the invention. The device includes a thin hollow element 1, such for instance a needle or a catheter, with a closed tip 2, suitable for penetrating the tissues to be subject to the TA procedure. The device is provided with an expandable and semipermeable membrane 3, wherein the hollow element 1 is coaxially inserted and sealed. In this particular embodiment, the hollow element 1 is made of a conductive material and it is connected to a radiofrequency energy generator. Thus in this embodiment the hollow element 1 is the active electrode of the TA device. The hollow element 1 is provided with one or more openings 4 circumferentially arranged in proximity of its tip 2. The end of the hollow element 1 is also surrounded by the membrane 3 that is sealed thereon. The residual portion of the hollow element 1 can be insulated, for example, by means of an insulating paint or an insulating sheath 5. Once inserted the hollow element 1 in the tumoral mass, an injection system injects a substance 6 through the opening or openings 4 of the hollow element 1 into the membrane 3, the substance 6 expands the membrane 3 thus generating on the tissues a pressure being higher than the atmospheric one, and permeates there through, thus closely contacting the surrounding tumoral tissues. Then the generator delivers electromagnetic energy, thus causing ionic turbulence in the zone surrounding the element 1 and thereby resistive heat. The transmission of the energy to the tissues is carried out due the electric conductivity properties of the substance 6, which contacts the hollow element 1. All the tissues being comprised between the electrodes and the 60° C. isotherm undergo to a non-reversible coagulative necrosis. Non-reversible damages are associated to temperatures comprised between 46° C. and 60° C., whose entity is proportional to the time of exposure.

The substance 6 must be biocompatible and capable of maintaining a low coupling impedance between the active part of the device and the tumoral tissues even at high temperatures. In such a way a continuous energy delivery from the device to the tissues is granted. In fact, as it may be learnt from a co-pending PCT patent application in the name of the same applicant, the injection into the tumoral mass of an electrically conductive substance, being capable of maintaining hydrated the region surrounding the electrode even at very high temperatures and/or maintaining the impedance constantly low during the energy delivery, allows to extend such a delivery for a very long time and thereby to generate thermal lesions having a large size without reaching the dehydration and the carbonization of the same tissues. Thereby it is possible to predict the size of the thermal lesion by setting a suitable time-profile of the power delivery.

Still in the co-pending PCT patent application, it may be learnt that the substance 6 is biocompatible, dehydrates or boils at temperatures being higher than the boiling temperature of the tissue liquids, has a viscosity being higher than that of the blood and has an electric conductivity comprised between one tenth and one hundred times the electric conductivity of the tissue liquids. The substance 6 may be a gel, a hydrogel, a thixotropic hydrogel, an aqueous ionic solution, a suspension having a size of the suspended particles comprised between about 1 μm and about 1000 μm, or a mixture of such substances.

One of the main characteristics of the invention is that the retaining action of the membrane 3 allows to keep the distribution of the substance 6 through the tissues totally under control, the substance permeating through the membrane 3 reaching the external surface thereof thus closely contacting the surrounding tissues. The possibility of exactly controlling the distribution of the substance 6, allows to surely predict the shape of the generated thermal lesion. The membrane 3 may have any shape, however in the preferred embodiments a cylindrical geometry is used with suitable zones connecting it to the hollow element 1.

Suitable materials for the manufacturing of the semipermeable membrane are, for example, the biological membranes, or woven or non-woven polymeric materials based on PET, PP, PA or PE.

Another characteristic of the device according to the present invention is that due to the effect of the injection of the substance 6 into the membrane 3, the local pressure on the tissues increases over the atmospheric pressure. As it may be learnt from a second co-pending PCT patent application in the name of the same applicant, the boiling temperature increase in the tissue liquids, being due to the pressure locally exerted by an expandable membrane, allows to deliver more energy to the tissues and thereby to generate thermal lesions having dimensions that are larger than those obtainable with known techniques.

The pressure inside the membrane 3 can be measured, for instance, by means of a pressure transducer and controlled in a close loop in order to grant the maintenance of the pre-set conditions for the whole duration of the procedure.

In FIG. 2 another embodiment of the device for TA with RF is shown according to the present invention. The design of the device is completely analogous to that of the device shown in FIG. 1, however this embodiment provides for the use of a cooling circuit being inserted into the hollow element 1, allowing to keep under control the temperature of the hollow element 1 during the treatment. In fact, as it is known the flow of electrical current generates resistive heat and the temperature profile of the heated zone has the maximum values close to the hollow element 1. The temperature control combined with the use of the substance 6 supports the duration of the TA procedures of and further increases the possibilities of setting the time-profile of the power. In the shown embodiment, the cooling circuit is composed of a small diameter canalization 7 being coaxially inserted into the hollow element 1. A conventional pumping system circulates a cooling substance 8 in the canalization 7, absorbing heat from the end of the element 1 and releasing it by passing, for instance, through a heat exchanger and then returning towards the end of the hollow element 1. The arrows 9 indicate an hypothetical circulation direction of the cooling substance 8 inside the canalization 7.

In FIG. 3 still another embodiment of the device for the TA with RF is shown according to the present invention. The design of the element 1 and of the membrane 3 is analogous to that of the previous drawings, however in this case the openings 4 provided in the proximity of the tip 2 of the hollow element 1 have a large size in order to allow the extraction of one or more filiform electrodes 10 in the space comprised between the hollow element 1 and the membrane 3. The electrodes 10 improve the energy delivery distribution as they increase the electrode surface thus allowing to further increase the efficiency of delivery of electromagnetic energy.

FIG. 4 shows a further embodiment of the device for the TA with RF according to the present invention, being analogous to the one shown in FIG. 3. In this case the filiform electrodes 10 are extracted from the hollow element 1 at the outside of the membrane 3 and contact the tissues. In other embodiments (not shown) it is also possible to combine filiform electrodes 10 inside and outside the membrane 3.

FIG. 5 shows a further embodiment of the device for TA with RF according to the present invention, using a bipolar technique for the delivery of electromagnetic energy. The end of the hollow element 1 enclosed in the membrane 3 is divided into an upper zone 11 and a lower zone 12 by interposing a ring 13 being made of an insulating material and having diameter and thickness equal to the hollow element 1. The two upper 11 and lower 12 zones are connected to the two poles of the circuit and form the active electrode and the counter electrode, respectively. In a TA procedure the substance 6 is injected into the membrane 3 through the openings 4 of the hollow element 1 as previously described. When switching on the generator, electromagnetic field lines are generated going from one electrode to the other one by crossing the substance 6 and causing, as in the previous cases, ionic turbulence and consequent resistive heat.

FIG. 6 shows an embodiment of the device for the TA according to the present invention of a microwaves type, wherein, in the same way as in the previous embodiments, the hollow element 1 is provided with a membrane 3 and with one or more openings 4 circumferentially arranged in proximity of the tip 2 of the hollow element 1. In this embodiment, differently from the previous ones, inside the hollow element 1 a coaxial cable 14 is arranged, delivering electromagnetic energy in the microwaves range. In this case the hollow element 1 is formed by materials being transparent to the microwaves in order not to interfere with their propagation through the tissues.

A further characteristic of the device and the method according to the present invention is that, once completed the TA procedure, the membrane 3 can be left in situ, that is in the necrotized tissue mass. The possibility of leaving the membrane in situ leads to a remarkable simplification of the procedure, which only requires the extraction of the hollow element 1 from the patient's body once it is ended. This does not affect the patient's health, as the membrane material is absolutely biocompatible as well as the substance 6 used to expand it.

The detachment of the hollow element 1 from the membrane 3 occurs in correspondence to connection areas 15 provided on the hollow element 1 by applying a predetermined load. For instance connection and release of the membrane could be accomplished by a gluing with a pre-set releasing load, by screwing and unscrewing rotating the catheter body on threaded corresponding profiles, or by snapping.

By means of the above-described devices it is possible to perform the TA method according to the present invention, comprising the steps of:
a. inserting a device into a tumoral mass, being provided with a hollow element 1 that is tightly inserted into an expandable membrane 3;
b. pressurizing said membrane 3 by injecting a substance 6; and
c. delivering electromagnetic energy at a high frequency in the tumoral mass till the coagulative necrosis of the tissues.
   The method provides for leaving the expandable membrane 3 in situ, that is inside the necrotized tissue, at the end of the TA treatment.

1.-24. (canceled)

25. A device for thermal ablation, comprising
a hollow element suitable for being connected to an electromagnetic energy generator at high frequency;
an expandable membrane made of biocompatible and semipermeable material and connected to said hollow element; and
a substance suitable for being injected into said expandable membrane through one or more openings provided on a portion of the hollow element connected to the membrane, the membrane being permeable to the substance,
wherein the substance is biocompatible, dries or boils at temperatures higher than a boiling temperature of tissue liquids, has a viscosity higher than blood viscosity and has an electric conductivity comprised between one tenth and one hundred times an electric conductivity of the tissue liquids.

26. The device of claim 25, wherein the membrane is made of a biological material.

27. The device of claim 25, wherein the membrane is made of a woven or non-woven fabric based on PET, PP, PA and/or PE.

28. The device of claim 25, wherein the substance is in the form of a gel.

29. The device of claim 25, wherein the substance is in the form of a hydrogel.

30. The device of claim 25, wherein the substance is in the form of a thixotropic hydrogel.

31. The device of claim 25, wherein the substance is in the form of an aqueous ionic solution.

32. The device of claim 25, wherein the substance is in the form of a suspension having a suspended particles size comprised between about 1 μm and about 1000 μm.

33. The device of claim 25, wherein the substance is a mixture of one or more substances chosen among a gel, a hydrogel, a thixotropic hydrogel, an aqueous ionic solution and a suspension having a suspended particles size comprised between about 1 μm and about 1000 μm.

34. The device of claim 25, further comprising transducers suitable for measuring pressure inside the membrane.

35. The device of one of claim 25, further comprising a cooling circuit formed of a small diameter canalization coaxially inserted into the hollow element and suitable for circulating a cooling substance.

36. The device of one of claim 25, further comprising one or more filiform electrodes extractable from the hollow element through said one or more openings.

37. The device of claim 25, further comprising one or more filiform electrodes extractable from the hollow element at the outside of the membrane.

38. The device of one of claim 25, wherein
an end of the hollow element is divided in an upper zone and a lower zone by a ring made of an insulating material and having diameter and thickness equal to those of the hollow element,
said upper and lower zones are respectively connected to the two poles of the circuit and
said membrane is coaxially assembled on the hollow element and sealed on the ring.

39. The device of one of claim 25, wherein the membrane is detachable from the hollow element in correspondence to connecting areas provided on the hollow element.

40. The device of claim 39, wherein the connecting areas between the membrane and the hollow element are made of a gluing having a pre-set releasing load.

41. The device of claim 39, wherein the connecting areas between the membrane and the hollow element are made of corresponding threaded profiles.

42. The device of claim 39, wherein the connecting areas between the membrane and the hollow element are made of a snapping mechanism.

43. A method for thermal ablation comprising the steps of:
providing a thermal ablation device according to claim 39;
inserting said device into a tumoral mass;
pressurizing the membrane by injecting the substance; and
delivering electromagnetic energy at a high frequency in the tumoral mass until coagulative necrosis of the tissues of the tumoral mass;
the method further comprising the step of leaving the membrane inside the necrotized tissue once thermal ablation is accomplished.

44. A method for thermal ablation comprising the steps of
providing a thermal ablation device according to claim 34;
inserting said device into a tumoral mass;
pressurizing the membrane by injecting the substance; and
delivering electromagnetic energy at a high frequency in the tumoral mass until the coagulative necrosis of the tissues of the tumoral mass,
the method further comprising the step of measuring and controlling the pressure inside the membrane.

45. The method of claim 44, wherein said measuring is performed through transducers.
